(19) United States
(12) Patent Application Publication
(10) Pub. No.: US 2015/0064142 A1
(43) Pub. Date: Mar. 5, 2015

(54) ELASTIC SCAFFOLDS FOR TISSUE GROWTH

(71) Applicant: Harvard Apparatus Regenerative Technology, Holliston, MA (US)

(72) Inventors: David Green, Dover, MA (US); Ron Sostek, Newton, MA (US)

(73) Assignee: Harvard Apparatus Regenerative Technology, Holliston, MA (US)

(21) Appl. No.: 14/394,464

(22) PCT Filed: Apr. 12, 2013
(86) PCT No.: PCT/US2013/036488

§ 371 Date: Oct. 14, 2014

Related U.S. Application Data
(60) Provisional application No. 61/623,548, filed on Apr. 12, 2012.
(60) Provisional application No. 61/624,229, filed on Apr. 13, 2012.
(60) Provisional application No. 61/636,600, filed on Apr. 20, 2012.

(51) Int. Cl. A61L 27/36; A61F 2/02

(52) U.S. Cl. CPC A61L 27/3604; A61F 2/02; A61F 2210/0057
USPC 424/93.7; 623/23.72; 427/2.24; 528/308.1; 118/621

(57) ABSTRACT

According to some aspects, tissue scaffolds are provided that comprise one or more types of nanofibers. In some embodiments, one or more design features are incorporated into a tissue scaffold (e.g., an electrospun tissue scaffold) to control the elasticity of the scaffold in at least one direction, making the scaffold suitable for withstanding mechanical forces when implanted in the body of a subject

### RELATED APPLICATIONS

This application claims priority under 35 U.S.C. §119(e) of U.S. Provisional Patent Application No. 61/623,548, filed on Apr. 12, 2012 and entitled “ELASTIC SCAFFOLDS FOR TISSUE GROWTH”; U.S. Provisional Patent Application No. 61/624,229, filed on Apr. 13, 2012 and entitled “ELASTIC SCAFFOLDS FOR TISSUE GROWTH”; U.S. Provisional Patent Application No. 61/636,600, filed on Apr. 20, 2012 and entitled “ELASTIC SCAFFOLDS FOR TISSUE GROWTH”. Each of these applications is incorporated herein by reference in its entirety for all purposes.

### BACKGROUND

Tissue engineering can involve generating a synthetic scaffold and seeding the scaffold to produce an engineered tissue that can be implanted into a subject. Different techniques have been used for producing synthetic scaffolds, including nanofiber assembly, casting, printing, physical spraying (e.g., using pumps and syringes), electrospinning, electrospraying, and other techniques for depositing one or more natural or synthetic polymers or fibers to form a scaffold having a suitable shape and size for transplanting into a subject (e.g., a human subject, for example, in need of a tissue or organ transplant).

Electrospinning and electrospraying techniques involve using a high voltage electric field to charge a polymer solution (or melt) that is delivered through a nozzle (e.g., as a jet of polymer solution) and deposited on a target surface. The target surface can be the surface of a static plate, a rotating drum (e.g., mandrel), or other form of collector surface that is both electrically conductive and electrically grounded so that the charged polymer solution is drawn towards the surface.

The electric field employed is typically on the order of several kV, and the distance between the nozzle and the target surface is usually several cm or more. The solvent of the polymer solution evaporates (at least partially) between leaving the nozzle and reaching the target surface. This results in the deposition of polymer fibers on the surface. Typical fiber diameters range from several nanometers to several microns. The relative orientation of the fibers can be affected by the movement of the target surface relative to the nozzle. For example, if the target surface is the surface of a rotating mandrel, the fibers will align (at least partially) on the surface in the direction of rotation. In some cases, the nozzle can be scanned back and forth between both ends of a rotating mandrel. This can produce a mesh of fibers that forms a cylinder covering at least a portion of the surface of the mandrel.

The size and density of the polymer fibers, the extent of fiber alignment, and other physical characteristics of an electrospun material can be impacted by factors including, but not limited to, the nature of the polymer solution, the size of the nozzle, the electrical field, the distance between the nozzle and the target surface, the properties of the target surface, the relative movement (e.g., distance and/or speed) between the nozzle and the target surface, and other factors that can affect solvent evaporation and polymer deposition.

Electrospun material can be used for a variety of applications, including as a scaffold for tissue engineering.

### SUMMARY

Tissue scaffolds are often subjected to mechanical forces (including stretching) when implanted in the body of a subject. This stretching is due to forces associated with physiological functions such as breathing and cardiovascular activity, in addition to normal movement and activity of the subject. In some embodiments, one or more design features are incorporated into a tissue scaffold (e.g., an electrospun tissue scaffold) to increase the elasticity of the scaffold in at least one direction relative to the underlying elasticity of the fibers or polymers that are used to form the scaffold.

According to some aspects, tissue scaffolds are provided that comprise one or more types of fibers (e.g., nanofibers) and that are elastic in a one or more directions. In some embodiments, the scaffold extends by 10-20% upon the application of about 5N of force in the one or more direction. In some embodiments, the scaffold extends by 20-40% upon the application of more than about 20N of force in the one or more directions. In some embodiments, the scaffold extends by 20-40% upon the application of more than about 40N of force in the one or more directions. In some embodiments, the scaffold extends by 20-40% upon the application of more than about 60N of force in the one or more directions. In some embodiments, the scaffold extends by 20-40% upon the application of more than about 80-100N of force in the one or more directions.

In some embodiments, the scaffold can extend by up to 100% in the one or more directions without experiencing structural failure. In some embodiments, the scaffold can extend by up to 150% in the one or more directions without experiencing structural failure. In some embodiments, the scaffold can extend by over 150% in the one or more directions without experiencing structural failure. In some embodiments, the one or more types of nanofiber include at least one electrospun nanofiber. In some embodiments, the at least one electrospun nanofiber is PET (polyethylene terephthalate). In some embodiments, the at least one electrospun nanofiber is PU (polyurethane). In some embodiments, a combination of PET and PU can be electrospun and/or electrosprayed as described herein.

In some embodiments, the one or more types of nanofiber include a nanofiber having a diameter of about 10-500 nm. In some embodiments, the one or more types of nanofiber include a nanofiber having a diameter of about 200-400 nm. In some embodiments, the one or more types of nanofiber include a nanofiber having a diameter of about 300 nm. In some embodiments, the one or more types of nanofiber have a density that provides pore spaces of 1-100 microns. In some embodiments, the one or more types of nanofiber have a density that provides pore spaces of about 50 microns.

In some embodiments, the scaffold is cellularized with one or more cell types. In some embodiments, the one or more cell types are obtained from a host into which the scaffold is to be implanted. In some embodiments, the one or more cell types are stem or progenitor cells. In some embodiments, the host is a human host. In some embodiments, the scaffold is tubular. In some embodiments, the scaffold has the shape and size of a human tracheal region. In some embodiments, the scaffold is branched. In some embodiments, the scaffold is elastic along the linear axis of the tubular shape.

According to some aspects of the invention methods of producing an elastic tissue scaffold are provided. In some embodiments, the methods comprise depositing one or more nanofiber types on to an elastic template. In some embodiments, the elastic template is manufactured from an elastic polymer. In some embodiments, the elastic template is hollow. In some embodiments, the elastic template is tubular. In some embodiments, the elastic template is planar. In some embodiments, the elastic template is shaped like a tissue, organ, or portion thereof. In some embodiments, the one or more nanofiber types are deposited by electrospinning. In some embodiments, the one or more nanofiber types include a PET or PU nanofiber. In some embodiments, the thickness of each of the one or more nanofiber types is between about 10 nm and about 500 nm. In some embodiments, the one or more nanofiber types have a density that provides for pore sizes of about 1-100 microns.

In some embodiments, methods of producing an elastic tissue scaffold are provided that comprise depositing one or more nanofiber types on a solid support, wherein the one or more nanofiber types are deposited in a pattern that allows a plurality of nanofibers to move relative to each other to allow the scaffold to be stretched in at least one direction. In some embodiments, the pattern is a woven pattern, a cross-hatched pattern, a net patterns, or other regular pattern of intersecting fibers. In some embodiments, the solid support is shaped like a tissue, organ, or portion thereof. In some embodiments, the one or more nanofiber types are deposited by electrospinning. In some embodiments, the one or more nanofiber types include a PET nanofiber. In some embodiments, the thickness of each of the one or more nanofiber types is between about 10 nm and about 500 nm. In some embodiments, the one or more nanofiber types have a density that provides for pore sizes of about 1-100 microns. In some embodiments, methods of producing an elastic tissue scaffold are provided that comprise depositing one or more nanofiber types on a solid support, wherein the one or more nanofiber types are deposited in a folded or coiled configuration that can be extended upon the application of a force, thereby allowing the scaffold to be stretched in at least one direction. In some embodiments, methods of producing an elastic tissue scaffold are provided that comprise depositing one or more nanofiber types on a solid support under conditions to impart a curvature on the one or more nanofiber types, wherein the curvature can be straightened upon the application of a force, thereby allowing the scaffold to be stretched in at least one direction. In some embodiments, the solid support is shaped like a tissue, organ, or portion thereof. In some embodiments, the one or more nanofiber types are deposited by electrospinning. In some embodiments, the one or more nanofiber types include a PET nanofiber. In some embodiments, the thickness of each of the one or more nanofiber types is between about 10 nm and about 500 nm. In some embodiments, the one or more nanofiber types have a density that provides for pore sizes of about 1-100 microns. In some embodiments, methods of producing an elastic tissue scaffold further comprise sterilizing the elastic scaffold.

In some embodiments, methods of producing an elastic tissue scaffold further comprise cellularizing the elastic scaffold. In some embodiments, an elastic scaffold produced according to the methods provided herein are implanted into a host. In some embodiments, the host is an animal. In some embodiments, the host is human. In some embodiments, a diseased or injured tissue is being replaced. In some embodiments, the diseased tissue is cancerous. In some embodiments, the nanofibers are deposited by vibration of the support or nozzle, wherein the vibration is sufficient to create a nanofiber pattern. In some embodiments, the nanofiber pattern is folded or wavy. In some embodiments, the tissue can withstand greater than 10% strain without failure. In some embodiments, the tissue can withstand greater than 20% strain without failure. In some embodiments, the tissue can withstand greater than 30% strain without failure.

According to some aspects, devices are provided for generating a synthetic tissue scaffold. In some embodiments, the devices comprise a collector; and an electrospray or electrospinning device configured and arranged for depositing a synthetic material on the collector. In some embodiments, the devices comprise a collector and a printer device configured and arranged for depositing cells and/or a synthetic material on the collector. In some embodiments, the devices comprise a collector; an electrospray or electrospinning device configured and arranged for depositing a synthetic material on the collector; and a printer device configured and arranged for depositing cells and/or a synthetic material on the collector.

### BRIEF DESCRIPTION OF DRAWINGS

FIG. 1 is a schematic drawing of an electrospinning device;

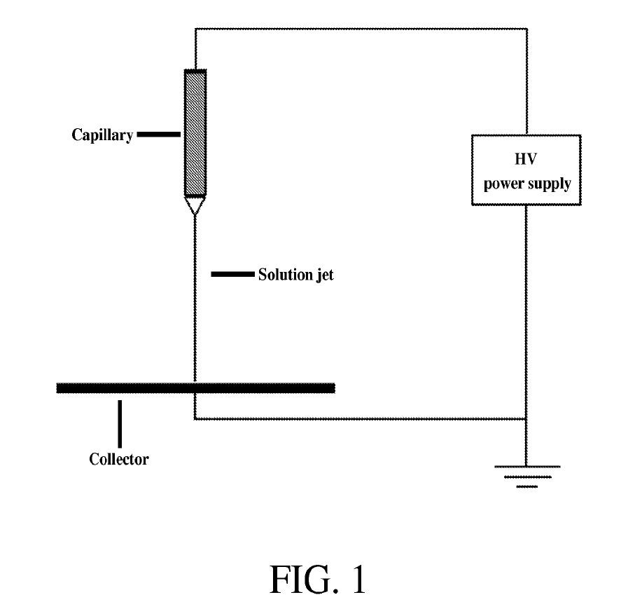

FIG. 2 is a schematic drawing of an electrospinning device having a vibrating nozzle configured for depositing;

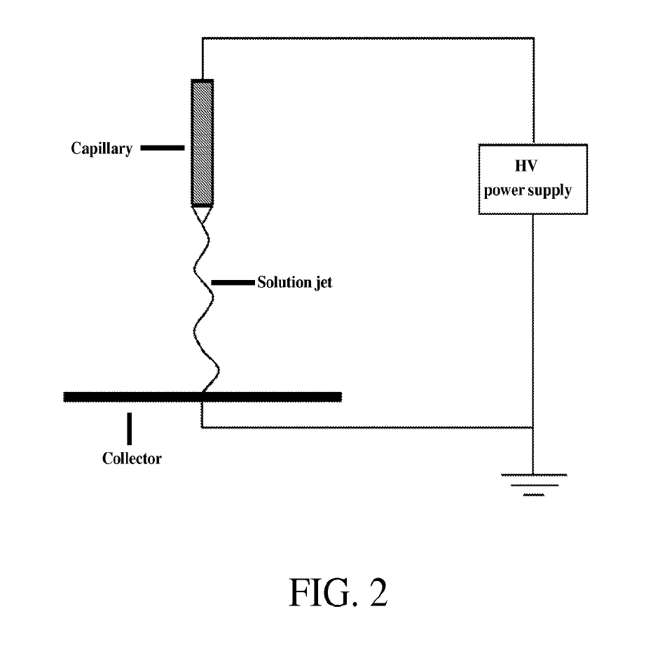

FIG. 3 illustrates a non-limiting embodiment of a fiber pattern that can be stretched elastically in a particular direction;

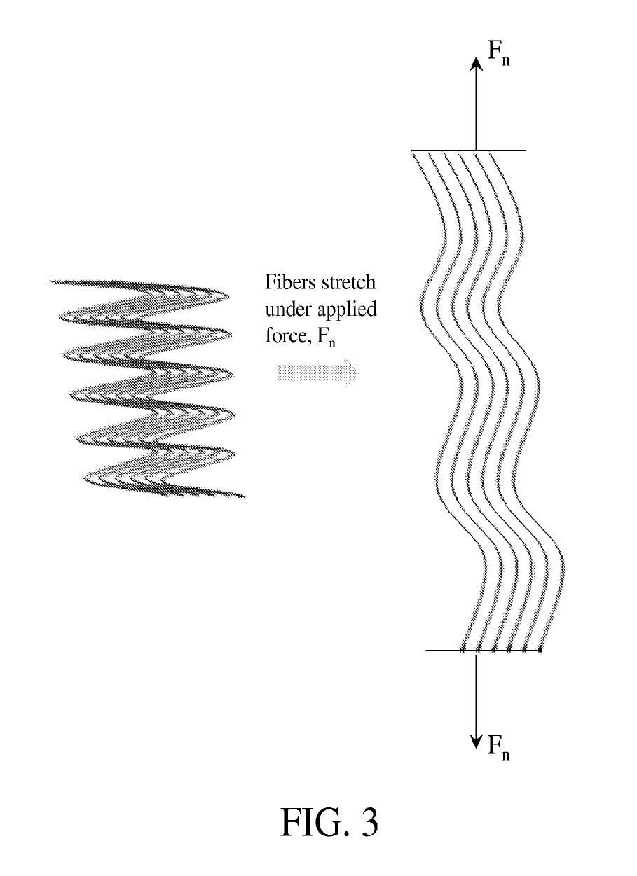

FIG. 4 illustrates a non-limiting embodiment of a cylindrical scaffold that can be stretched along its length;

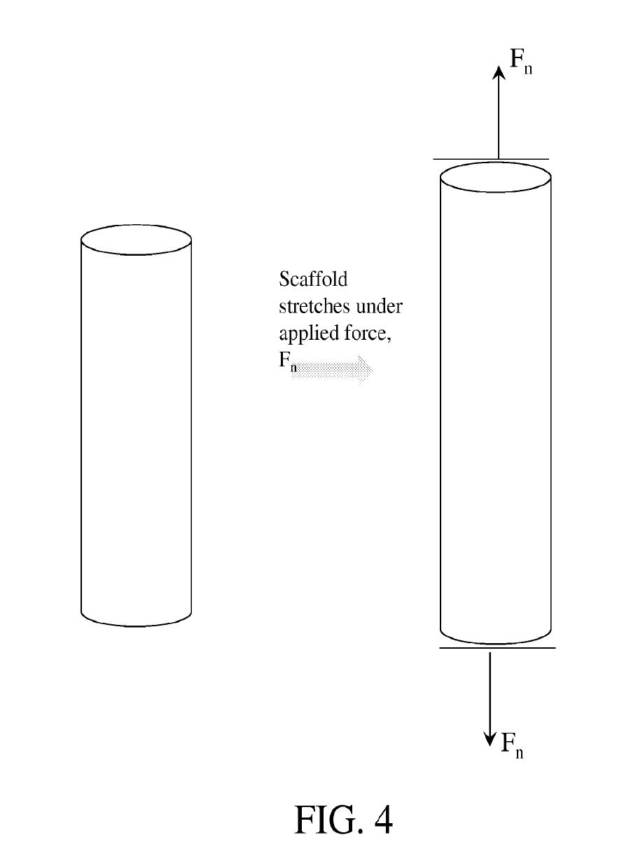

FIG. 5 illustrates a non-limiting embodiment of a fiber delivery system; and

FIG. 6 illustrates non-limiting embodiments of fiber mixing systems.

### DETAILED DESCRIPTION OF CERTAIN EMBODIMENTS

In some embodiments, aspects of the invention relate to methods, compositions, and articles for producing artificial (e.g., synthetic) tissues, organs, or portions thereof that can be implanted into a host (e.g., a human host) to replace diseased or injured tissues, organs, or portions thereof.

In some embodiments, aspects of the invention relate to scaffolds that are used for tissue growth, and that are sufficiently elastic to undergo physiological levels of strain without breaking. Scaffolds generated as described herein can be seeded with appropriate cell types to produce artificial tissues or organs or portions thereof for transplantation into a host.

In some embodiments, aspects of the invention relate to elastic scaffolds, for example scaffolds that can undergo at least about 10% (e.g., at least about 20%, at least about 30%, at least about 40%, at least about 50%, at least about 60%, at least about 70%, at least about 80%, at least about 90%, at least about 100%, or more) strain (e.g., tensile strain) in one or more directions without mechanical failure (e.g., breaking). In some embodiments, the scaffold forms a hollow cylinder that can undergo at least about 10% (e.g., at least about 20%, at least about 30%, at least about 40%, at least about 50%, at least about 60%, at least about 70%, at least about 80%, at least about 90%, at least about 100%, or more) tensile strain in a longitudinal direction without mechanical failure (e.g., breaking). In some embodiments, the scaffold forms a hollow cylinder that can undergo at least about 10% (e.g., at least about 20%, at least about 30%, at least about 40%, at least about 50%, at least about 60%, at least about 70%, at least about 80%, at least about 90%, at least about 100%, or more) hoop tensile strain without mechanical failure (e.g., breaking).

In some embodiments, elastic scaffolds comprise one or more types of fiber (e.g., nanofibers). In some embodiments, elastic scaffolds comprise one or more natural fibers, one or more synthetic fibers, one or more polymers, or any combination thereof.

Aspects of the invention are useful for producing scaffolds that are more elastic than current scaffolds used for tissue growth. Scaffold elasticity can be important for producing artificial tissues or organs having sufficient elasticity to withstand the physical demands at the site of implantation. It should be appreciated that tissue elasticity can be important for many different tissues, including respiratory tissues (e.g., tracheal, bronchial, esophageal, alveolar, and other pulmonary or respiratory tissues), circulatory tissues (e.g., arterial, venous, capillary, and other cardiovascular tissue), renal tissue, liver tissue, cartilaginous tissue (e.g. nasal or auricular), skin tissue, and any other tissue that may benefit from elasticity at the site of implantation in a host.

In some embodiments, scaffold or tissue elasticity is a measure of the extent to which the scaffold or tissue can be extended or stretched (e.g., from a resting or stable state, e.g., prior to implantation or at the site of implantation) in response to the application of a force across that scaffold or tissue direction (e.g., exerting force by pulling on opposite ends of the structure along one or more axes of the structure). In some embodiments, an elastic scaffold or artificial tissue will extend or stretch by up to 20% (e.g., more than 10%, for example 10-20%, or about 15%) in one or more directions upon the application of a moderate force, for example, about 1-10 Newtons, about 3-7 Newtons, about 4 Newtons, about 5 Newtons, or about 6 Newtons. In some embodiments, an elastic scaffold or artificial tissue will extend or stretch from 20-40% in one or more directions upon the application of additional force, for example on the order of 20-140 Newtons, about 40-120 Newtons, or about 80-100 Newtons. In some embodiments, elastic scaffolds or artificial tissues should be able to undergo over 60% (e.g., up to 100%, up to 120%, up to 140% or up to 150%, or more) extension in one or more directions without sustaining a structural failure (e.g., plastic deformation or tearing). In some embodiments, the force is applied over physiological time frames, e.g., up to 30 seconds, up to 1 minute, up to 2 minutes, up to 5 minutes, or up to 10 minutes. It should be appreciated that in some embodiments an elastic scaffold or artificial tissue returns to its resting or stable size upon removal of the external force. Depending on the type or tissue or organ that is being replaced, the scaffold or artificial tissue may be designed and manufactured to have different elasticity profiles. For example, respiratory tissues are generally subject to more stretching during their normal function than organs such as liver or kidneys. Therefore, scaffolds for respiratory tissues may need to be more elastic than scaffolds for other organs such as livers and kidneys. Nonetheless, liver and kidney tissue do require some degree of elasticity for optimal function. It should be appreciated that there is not necessarily a linear relationship between the amount of extension and the applied force. In some embodiments, an initial % extension can be achieved with relatively little force, but further extension requires significantly more force. In some embodiments, for tracheal replacements, the range of elasticity should be about 20-40% elastic extension under natural biological conditions (at least in one direction, for example along the long axis). For example, about 20% extension should occur in at least one direction (for example along the long axis) with a load of around 4 Newtons, whereas the 20-40% stretch should occur with a load of about 80-100 N.

It should be appreciated that the elasticity of a scaffold or artificial tissue does not have to be the same or similar in different directions. For example, certain tissues (e.g., certain airway tissues) may extend more in a linear direction than in a radial direction. For example, a 10 cm length of an approximately tubular tracheal replacement scaffold or tissue (e.g., having a diameter of 1-2 cm) may extend to about 12 cm in length upon the application of about 4 Ns along the linear axis, and would further extend to about 14 cm upon the application of about 80-100N. In contrast, the diameter may not change as much in response to the same forces. However, it should be appreciated that the relative elasticity of scaffolds and artificial tissues can be different for different physiological tissues and applications. The relative elasticity in different directions can be adjusted by the design of the scaffold, for example, by including different structural components, thicknesses, material, etc., or any combination thereof, in different patterns along different directions. For example, an airway replacement can be maintained relatively rigid in the radial direction by including one or more supporting ribs. This can still allow for suitable elasticity in the long axis by incorporating elastic scaffold or artificial tissue in the regions between the ribs. In addition, it should be appreciated that the degree and profile of scaffold or tissue elasticity in different directions can be adjusted by modifying one or more of the parameters described herein in order to obtain physiologically appropriate two dimensional or three dimensional elasticity.

In some embodiments, elastic scaffolds are formed as tubular structures that can be seeded with cells to form tubular tissue regions (e.g., tracheal, bronchial, or other tubular regions). It should be appreciated that a tubular region can be a cylinder with a uniform diameter. However, in some embodiments, a tubular region can have any appropriate tubular shape (for example, including portions with different diameters along the length of the tubular region). A tubular region also can include a branch or a series of branches. In some embodiments, an elastic tubular scaffold is produced having an opening at one end, both ends, or a plurality of ends (e.g., in the case of a branched scaffold). However, elastic tubular scaffold may be closed at one, both, or all ends, as aspects of the invention are not limited in this respect. It also should be appreciated that aspects of the invention may be used to produce elastic scaffolds for any type or organ, including hollow and solid organs, as the invention is not limited in this respect.

In some embodiments, a scaffold is produced using a support (e.g., a solid or hollow support) on which the scaffold can be formed. For example, a support can be a mandrel, tube, or any other shaped support. It should be appreciated that the support can have any size or shape. However, in some embodiments, the size and shape of the support is designed to produce a scaffold that will support an artificial tissue of the same or similar size as the tissue being replaced or supplemented in a host (e.g., trachea or other airway portion, blood vessel, liver or kidney region, or other tissue or organ).

In some embodiments, an elastic scaffold can be produced by depositing fibers on an elastic template (e.g., a stretchable macroscale fabric). An elastic template can be an elastic material that is placed over a support. For example, an elastic template can resemble a sock or sheath or other covering that is placed over a mandrel. However, it should be appreciated that any suitable shape of elastic template can be used (e.g., a sheet, a strip, a cylinder, whether regular or irregular, or any other suitable shape). In some embodiments, the elastic template is placed over a shaped support, for example a conducting support that can be used for depositing electrospun fibers onto the template. However, other types of fibers can be deposited. Also, in some embodiments, the elastic template does not need to be placed on a shaped support (e.g., it could be placed on a surface (e.g., a planar or curved surface), for example a conducting surface, or in a solution, or hanging on a support, or in any other suitable configuration). In some embodiments, one or more types of nanofibers are deposited on the support using electrospinning as described herein. However, other types of fibers can be deposited on the elastic template, in addition to or instead of the electrospun fibers, as aspects of the invention are not limited in this respect. In some embodiments, one or more polymers or fibers may be printed onto a template, electrospun onto a template, or both.

In some embodiments, the elastic properties of the elastic template are selected to be similar to the elastic properties of the tissue or organ or portion thereof that is being replaced. It should be appreciated that the fibers and/or cells that are added to the elastic template may change the elastic properties. Accordingly, the elastic properties of the template may be selected so that the elastic properties of the final artificial tissue or organ is similar to the host site being replaced or supplemented. However, it also should be appreciated that the elastic properties of the artificial tissue do not need to be identical to those of the host region, provided that the elastic properties are sufficient to provide beneficial structural and functional properties when implanted into the host (e.g., human host).

In some embodiments, the elastic template may consist of or include one or more of the following materials: elastic polymers (e.g., one or more polyurethanes, for example polycarbonates and/or polyesters), Nylon, resorbable materials (e.g., PLGA, PLA, PGA, PCL), synthetic or natural materials (e.g., silk, elastin, collagen, etc.) or any combination thereof. In some embodiments, the elastic template may consist of or include addition polymer and/or condensation polymer materials such as polyolefin, polyacetal, polyamide, polyester, cellulose ether and ester, polyalkylene sulfide, polyarylene oxide, polysulfone, modified polysulfone polymers and mixtures thereof. In some embodiments, the elastic template may consist of or include polyethylene, polypropylene, poly(vinylchloride), polymethylmethacrylate (and other acrylic resins), polystyrene, and copolymers thereof (including ABA type block copolymers), poly(vinylidene fluoride), poly(vinylidene chloride), polyvinylalcohol in various degrees of hydrolysis (e.g., 87% to 99.5%) in crosslinked and non-crosslinked forms. In some embodiments, the elastic template may consist of or include block copolymers. In some embodiments, addition polymers like polyvinylidene fluoride, syndiotactic polystyrene, copolymer of vinylidene fluoride and hexafluoropropylene, polyvinyl alcohol, polyvinyl acetate, amorphous addition polymers, such as poly(acrylonitrile) and its copolymers with acrylic acid and methacrylates, polystyrene, poly(vinyl chloride) and its various copolymers, poly(methyl methacrylate) and its various copolymers, can be solution spun in producing an elastic template. In some embodiments, highly crystalline polymers like polyethylene and polypropylene may be solution spun in producing an elastic template.

In some embodiments, the elastic template is sufficiently thin and/or sparse to avoid interfering with an electrical deposition technique (e.g., electrospinning or electro spraying). In some embodiments, the elastic template can include one or more electrically conductive materials so that the elastic template also is conductive and allows an electrical deposition technique to proceed. Non-limiting examples of electrically conductive materials that can be incorporated into an elastic template include conductive metals (e.g., silver, copper, annealed copper, gold, aluminum, calcium, tungsten, zinc, nickel, lithium, iron, platinum, tin, lead, titanium, manganin, constantan, mercury, nichrome, carbon (amorphous)); conductive plastics; conductive or anti-static powders/agents (e.g., the EP1/EP2/EP3/EP4 series available commercially from Noelson Chemicals); conductive glass powder (e.g., the EG series available commercially from Noelson Chemicals); conductive mica powder (e.g., the EC-300 series available commercially from Noelson Chemicals); conductive titanium dioxide (e.g., EC-320 series available commercially from Noelson Chemicals); conductive barium sulfate (e.g., the EC-340 series available commercially from Noelson Chemicals); conductive ATO powder (e.g., the EC-360 series available commercially from Noelson Chemicals); conductive zinc oxide (e.g., the EC-400 series available commercially from Noelson Chemicals); conductive polyaniline (e.g., the EC-600 series available commercially from Noelson Chemicals); conductive carbon or black/conductive graphite (e.g., the EC-380/EC-390 series available commercially from Noelson Chemicals); high conductive carbon powder (e.g., the EC series available commercially from Noelson Chemicals), and/or carbon nanotubes (e.g., the EC-700 series available commercially from Noelson Chemicals).

In some embodiments, elastic scaffolds can be produced without using an elastic template.

In some embodiments, a fiber-based scaffold can be deposited directly on a support (e.g., a shaped support as described herein) in a pattern that provides elastic properties even if the types of fibers that are used are not very elastic. In some embodiments, a structured elastic scaffold can be generated using electrospinning, electrospraying, physical spraying, printing, or a combination thereof by depositing appropriate patterns on the support (e.g., “spray painting” using any suitable deposition technique to produce specific patterns on the support). For example, one or more strips of relatively dense fibers can be deposited in a cross hatched pattern (e.g., to form a net-like or chain-link-like pattern on the support). Such a pattern could be similar to the structure of a woven or knitted fabric, a knotted fabric, a net, or other crossed pattern. It should be appreciated that the strips can be deposited with different thicknesses, different widths, different densities, or at different relative angles, or any combination thereof. These different factors can be used to tune the elasticity of the resulting scaffold using one or more types of fibers. Accordingly, elastic structures can be formed from relatively inelastic materials (e.g., PET). As described herein, appropriate patterns of elasticity are different for different tissues and can be obtained by adjusting the patterns and thicknesses of the different fibers that are used.

In some embodiments, an elastic fiber-based scaffold can be formed by generating fibers that are folded or coiled (or otherwise compacted) before or during their deposition on a support (e.g., a mandrel). In some embodiments, the nozzle (either a single nozzle or an array of nozzles) of a syringe that is delivering a fiber (e.g., for an electrospun fiber) can be rotated during deposition (e.g., during spinning) to create coiled fibers. In some embodiments, the support can be rotated relative to a fixed nozzle (either a single nozzle or an array of nozzles) during deposition (e.g., during spinning) to create coiled fibers. In some embodiments, the relative positions of the nozzle and support can be moved in other ways (e.g., vibrated, etc.) during deposition to create other types of two-dimensional or three-dimensional fiber structures (e.g., folds) that can provide elastic properties. In some embodiments, the nozzle is vibrated (or otherwise moved relative to the support) during deposition to a sufficient degree to create a folded or wavy pattern of fibers. In some embodiments, the support is vibrated (or otherwise moved relative to the nozzle) during deposition to a sufficient degree to create a folded or wavy pattern of fibers. It should be appreciated that the frequency and amplitude of the vibration (or other movement) of the support affects the pattern (e.g., two-dimensional or three-dimensional pattern) of the fiber. This is in turn affects the extent to which the fiber can be extended or stretched in one direction before reaching its maximal length (and ultimately breaking if sufficient force is applied). In some embodiments, other physical forces (e.g., pressure waves, ultrasound, etc.) can be applied to form folds or other three-dimensional fiber structures during deposition.

The nozzle or support may be suitably fitted with one or more piezoelectric actuators, magnetostrictive actuators, or other suitable actuators, that is/are configured to control the amplitude, direction and/or frequency of vibration or oscillation of the nozzle and/or support relative to one another, thereby controlling the pattern of which polymers are laid or layered onto the support. In some embodiments, the frequency of vibration is up to 500 hertz (Hz), up to 1 kHz, up to 10 kHz, up to 100 kHz, up to 1 MHz, or more. In some embodiments, the frequency of vibration is 10 Hz to 500 Hz, 10 Hz to 1000 Hz, 100 Hz to 1000 Hz, 500 Hz to 1 kHz, or 1 kHz to 1 MHz. In some embodiments, the amplitude is constant. In some embodiments, the amplitude of vibration is variable. In some embodiments, the amplitude of vibration varies in a non-random manner. In some embodiments, the amplitude of vibration varies in a random or pseudo-random manner. In some embodiments, the amplitude of vibration of the nozzle or support is in a range of 1 nm to 100 nm, 1 nm to 500 nm, 10 nm to 500 nm, 100 nm to 1 μm, 500 nm to 10 μm, 1 μm to 10 μm, or 1 μm to 100 μm. In some embodiments, the amplitude of vibration of the nozzle or support is 1.5-fold, 2-fold, 3-fold, 4-fold, 5-fold, 10-fold or more than the diameter of the nanofibers being laid.

In some embodiments, a two-dimensional and/or three dimensional curvature can be imparted on a fiber by stretching one side or surface of the fiber relative to the other (for example like a curved ribbon). In some embodiments, this can be achieved by passing a fiber through an atmosphere with a velocity gradient. In some embodiments, this can be achieved by co-extruding two or more different polymers or other material (and/or two or more concentrations of the same polymer or other material) where one of the polymers (or other material) shrinks or expands more than another after extrusion. For example, one polymer can shrink slightly upon solvent evaporation relative to the other, thereby producing a shortening on side of the fiber relative to the other.

In some embodiments, a polymer that is cured or partially cured by exposure to a particular condition (e.g., UV radiation, heat, chemical reagent, humidity, other temperature change, solvent concentration in the air, or other condition) can be shaped (e.g., curved or bent) to produce elastic properties. For example, by exposing a fiber stream to the curing condition from one side or surface, that side or surface will cure faster relative to the other, leading to coiling of the fibers prior to or during deposition.

In some embodiments, macroscale structures (e.g., yarns) can be produced from nanoscale or microscale fibers (e.g., much like wool is twisted into threads from fibers taken from sheep). The macro scale structures can be woven or knitted to form an elastic fabric having spaces between the macroscale fibers. In some embodiments, the spaces can be relatively small (the elastic fabric is relatively tightly knit). In some embodiments, the inner structure of the macroscale threads retains a nanoscale environment suitable for cellular growth while maintaining the more elastic properties of the macroscale weave or knit.

Various methods known in the art may be used to produce such nanoscale fibers (nanofibers). In some embodiments, nanofibers may be produced using techniques such as template synthesis, phase separation, self-assembly or electrospinning. In some embodiments, template synthesis involves extruding a polymer solution through nanopores (e.g., a membrane with nanoscale pores) to produce extruded nanofibers. In some embodiments, phase separation involves mixing a polymer with a solvent under conditions in which gelation of the polymer occurs, and following gelation, extracting the solvent leaving behind a porous nanostructure. In some embodiments, self-assembly of nanofibers refers to the growth of nanoscale fibers using smaller molecules as basic building blocks. In some embodiments, electrospinning may be used to produce fibers (e.g., randomly orientated, aligned, patterned) with essentially any chemistry and a wide range of diameters (e.g., diameters ranging from 15 nm to 10 μm). In some embodiments, the substrate upon which deposition takes place during an electrospinning is conductive in order to attract a falling fiber out of the air. Additional techniques for nanofiber formation include electrospinning a polymer-containing solution or a polymer melt onto a rotating substrate held at a different potentials than the solution spray nozzle to form nanofibers,

It should be appreciated that these techniques described herein may be used alone or in combination. It also should be appreciated that the following techniques also may be used, either alone or in combination (for example in combination with each other or with the techniques described above) to produce scaffolds having suitable elastic properties.

In some embodiments, the electrical field strength can be varied along the fiber axis while it is in the air so as to stretch and relax the fiber while it is being spun. In some embodiments, two or more nozzles from two or more different syringes at different angles of incidence relative to the support (e.g., relative to an axis of a shaped support, for example, the longitudinal axis of the mandrel) can be used to adjust tension on the fibers as they are deposited. In some embodiments, alternating layers of material along a surface of a support (e.g., along the axis of a tube), for example in a repeating pattern, e.g., “ab ab ab ab” where “a” has a different length to “b”, and/or “a” is a different material to “b”. It should be appreciated that other patterns of different lengths, thicknesses, and/or materials, also may be used to produce desirable elasticity of a scaffold. In some embodiments, one or more folds or other three-dimensional structures (e.g., in the form of a concertina, bellows, or other expandable and/or collapsible structures) can be introduced into the material to allow expansion and/or contraction. In some embodiments, material of different elasticity may be used for different parts of a scaffold. For example, a pattern (e.g., a slinky spiral or other pattern) of stronger sections may be used to provide elasticity. In some embodiments, elastic material may be used on one side (e.g., the inside of a tubular structure) and less elastic electrospun material may be used on the other side (e.g., on the outside). In some embodiments, the patterns of fibers can vary along the surface of the material such that different degrees of elasticity can occur at different positions along the same structure. In some embodiments, this is accomplished by mixing different polymers together or by alternating the pattern of fibers, or both.

In some embodiments, fibers (e.g., nanofibers) may be deposited on (e.g., electrospun onto) a stretched elastic template (e.g., stretched over a shaped support such as a mandrel) so that the scaffold compresses (at least partially) after removal from the support. In some embodiments, one or more fibers may be deposited (e.g., electrospun) in a pre-stretched form so that the scaffold shrinks when it is removed from the shaped support (e.g., the mandrel). In some embodiments, this can be achieved by rotating the support (e.g., mandrel) at a speed such that the linear speed of the surface of the support exceeds the linear speed of the fiber as it approaches the mandrel. In some embodiments, in order to provide elasticity (e.g., suitable stretching properties) in the longitudinal axis of the scaffold, the axial velocity of the support would need to be higher. In some embodiments, different patterns of deposition (e.g., fiber deposition in a spiral in one direction along the main axis followed by deposition of the fibers in a spiral in the opposite direction along the axis). In some embodiments, certain portions of a scaffold (e.g., one or more ribs, for example C-shaped or U-shaped ribs on an airway scaffold, for example a tracheal scaffold) may be porous (e.g., formed from a foamed material or other method of generating a porous structure) to provide suitable elasticity.

In some embodiments, high frequency changes of the mixture of two or more polymers can be used (e.g., mix changes in an “ab ab ab ab” pattern during the time of flight of the fibers to create S-shaped fibers rather than coiled fibers) to generate fiber shapes that can provide elasticity.

In some embodiments, synchronization of a rotation angle (or longitudinal position of a mandrel) of a support structure (e.g., mandrel) with flow rate/composition of ejection material can be used to allow deposition of different materials at adjustable sites on a rotating support (e.g., mandrel). By adjusting flow rate, distance, and speed of rotation of the support (e.g., mandrel) the position of the fiber being deposited on the support can be predicted, thereby allowing fiber patterns that provide suitable elasticity to be generated.

In some embodiments, support structures having different patterns of conductivity on their surface can be used to generate different patterns of fiber deposition. Accordingly, patterns that provide elastic properties (e.g., U-shaped, C-shaped, S-shaped, O-shaped, or other simple or complex shapes that can be compressed or stretched) can be deposited on a support to generate scaffolds that have desirable elastic properties.

In some embodiments, masks, solvent application, or other techniques can be used, alone or in combination with other techniques described herein, to produce desired fiber patterns. For example, in some embodiments, desired deposition patterns can be obtained by selectively directing fibers or polymers to particular locations on a support (e.g., collector) surface by varying the electric field strength at the surface of the support by selectively masking portions of the conducting surface with an insulator.

In some embodiments, desired deposition patterns can be obtained by selectively directing fibers or polymers to particular locations on a support (e.g., collector) surface by varying the electric field strength at the surface of the support by selectively activating or inactivating (e.g., electrically activating or inactivating) portions of the collector surface. In some embodiments, a collector (e.g., mandrel) has a plurality of locations at which the electric charge can be controlled. For example, in some embodiments a plurality of electric circuits can be included at or beneath the surface of a collector. This allows the electric field strength at the surface of the collector to be controlled thereby providing a set of addressable destinations for fibers or polymers that are being deposited by electrospinning or electrospraying. In some embodiments, one or more first regions can be made selectively conductive (while other regions are maintained in a non-conductive state) in order to promote fiber deposition in the conductive regions. In some embodiments, the electric field at a location at which deposition is desired can be set to have an opposite charge (and therefore be electrically attracting) of the charge of the electrically charged solution or melt that is being deposited. In some embodiments, the electric field at a location at which deposition is not desired can be set to have a same charge (and therefore be electrically repelling) as the charge of the electrically charged solution or melt that is being deposited.

In some embodiments, the conductivity and/or electrical voltage that is applied to particular locations on a collector (e.g., mandrel) surface can be directly controlled. For example, in some embodiments, the surface of a collector can be a thin insulating layer such as a polymer, underneath which an array of conductors (e.g., wires, 2-D shapes such as radial bands, or other conductive material) can be used to selectively modify the electric field at different locations so that fibers can be preferentially attracted to the conducting zones that are conductive and/or that have an attracting electric charge. It should be appreciated that this technique allows different types of polymers to be selectively deposited at different locations. In some embodiments, a first pattern of conductivity and/or electric charge is imposed on the collector when a first polymer or fiber solution is being deposited, and a second pattern of conductivity and/or electric charge is imposed when a second polymer or fiber solution is being deposited (or when deposition of more of the same first polymer or fiber is desired). For example, in some embodiments the electrically conductive and/or attracting area of a mandrel surface can be switched between a first pattern that includes essentially the entire surface of the mandrel and a second pattern that includes only a set of radial rings (e.g., evenly spaced along the axis of the mandrel). Initially, the entire surface can be appropriately activated and fibers are deposited evenly over the entire surface. After an initial layer of fiber is deposited on the mandrel surface (e.g., an approximately 1 mm thick layer), the pattern of radial rings can be selectively activated and fiber deposition can be continued resulting in preferential deposition at the locations of the activated radial rings. This creates a thicker layer of fiber at each of these positions. In some embodiments, these radial rings can correspond to a rib structure on a synthetic tracheal scaffold. In some embodiments, after each ring location has been strengthened by the deposition of additional fiber material (e.g., an accumulation of an additional mm or more of fiber thickness at each of the ring locations), the pattern of activation can be switched back to the entire mandrel surface in order to deposit a second layer of fiber over the entire surface. In some embodiments, this results in a scaffold that is more flexible (and/or elastic) in regions that are thinner (e.g., in the regions between the rings).

It should be appreciated, that collectors having different shapes and sizes (including static or rotating collectors) can be produced with appropriate circuits or conductor elements to allow selective activation of one or more different locations (or patterns) on the collector surface. This allows an electric field to be controlled over the 2 dimensional or 3 dimensional surface of a collector regardless of the shape of the collector. Accordingly, selective electrical activation and/or inactivation can be used to selectively deposit one or more materials at specified locations or in predetermined patterns over a flat surface (such as a square or circular plate collector), a tubular surface (such as a mandrel), or more a complex three dimensional surface (such as the branching structure of a lung) where it can be challenging to deposit fibers at the center of the scaffold (for example on the core trunk of the branching structure) due to the presence and interference of the outer fine structure of the scaffold (for example the outer fine branches of a branching structure). In some embodiments, one or more regions of a branching structure can be deactivated (e.g., electrically) by making those regions non-conductive or by imposing a repelling electrical field on those regions, without deactivating a target region of interest (e.g., a trunk of a branching structure) that is maintained in a conductive and/or electrically attracting state.

In some embodiments, certain elasticity can be provided by using different layers or thicknesses of material, for example by electrospinning a layer of PET fibers then adding solid strips of PET on top of the fiber layer to make the scaffold more rigid then spinning a second layer of fibers over the top of the solid strips. However, this composite manufacturing process can lead to sharp edges on the solid strips, weak joints between the layers and the solid strips (and this can lead to delamination and/or movement of the solid strip relative to the fibers). In some embodiments, the weakness of the joints and/or the movement of the solid strips can cause damage to the cells growing on the scaffold and/or to adjacent tissues in the body once the implant has taken place.

In some embodiments, fibers (e.g., nanofibers) may be “spray painted” (e.g., by electrospraying and/or by physical spraying, for example by generating an aerosol) onto a support (e.g., a mandrel) to achieve a very tight spatial control over the deposition of the fibers so that areas of greater and lesser density of fibers can be deposited. This can be achieved, for example, by making the distance from the syringe needle to the mandrel very small, electrically masking off the areas on the mandrel where fibers are not wanted. In some areas (e.g., the rib areas of an airway scaffold or where the ribs join the member which runs perpendicular to the ribs to make up the longitudinal edges of the scaffold, e.g., along the long edges of the tracheal/esophageal boundary) the density of the fibers can be made much higher making these parts almost solid. However, in some embodiments, because the process of deposition is continuous there will be no sudden density change (from fiber to solid material) and hence no sharp edges. In some embodiments, this “spray painting” of the fibers into defined locations on the support (e.g., mandrel) can be accomplished by either by human hand or using a shoulder/elbow/wrist robot that holds the syringe and needle (e.g., using a syringe pump under the brand name Nanomite, for instance, with the injector end mounted on the robot “hand”) to accurately deposit the fibers at the required locations and densities.

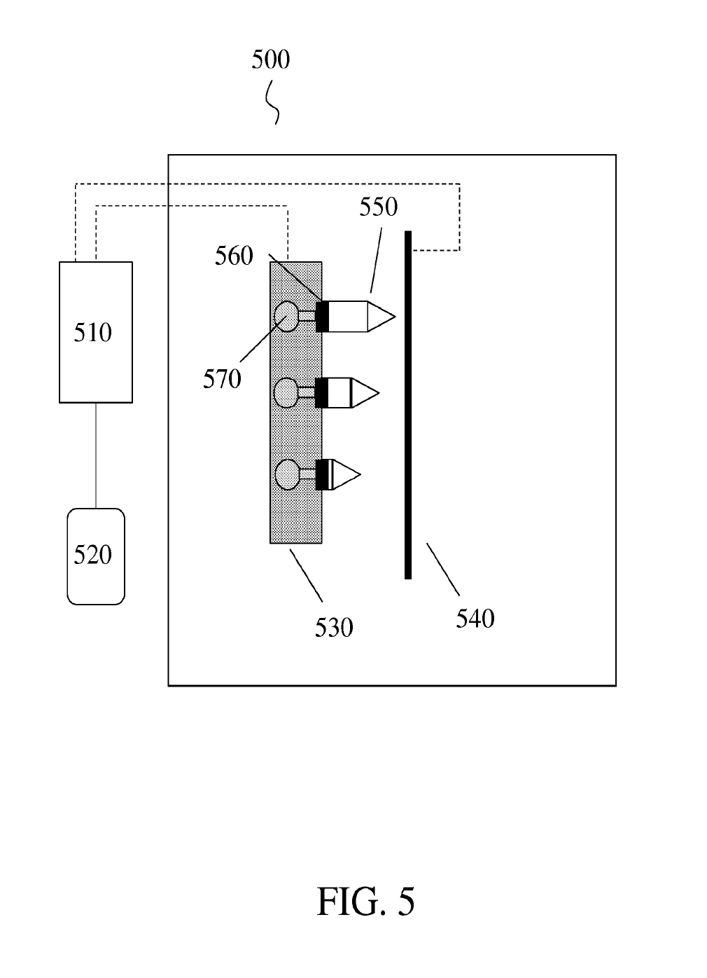

In some embodiments, polymers and/or fibers can be deposited by printing or electrospraying or electrospinning. In some embodiments, a device that can both print and electrospray or electrospin can be used. For example, a device may include a single needle or nozzle that is capable of printing or electrospraying/spinning nanofibers. However, a device may include, in some embodiments, at least two different needles or nozzles, one for printing and the other for electrospraying/spinning. In some embodiments, a combination of printing and/or electrospinning units can be used. In some embodiments, polymer or fiber deposition can be performed by printing and/or electrospinning units, wherein both functions are carried out in sequence or simultaneously with the same or two different systems working on the same elastic scaffold. FIG. 5 illustrates a non-limiting example of a polymer or fiber deposition system. In some embodiments, the system is housed within a chamber (500) that can be a vacuum chamber and/or an environmental chamber that can be used to specify and control the temperature, humidity, air flow, light exposure (e.g., UV exposure), and/or any other environmental parameter that can be used to affect polymer or fiber deposition. Accordingly, in some embodiments, chamber (500) can include or be connected to one or more heating elements, cooling elements, humidifiers, dehumidifiers, light sources (e.g., UV sources), or other devices or means that can be used to control the environment of the deposition system. However, it should be appreciated that in some embodiments, a deposition system is not enclosed within a chamber (500) as aspects of the invention are not limited in this respect. In some embodiments, a polymer or fiber deposition system includes a high-voltage power source (510), a controller (520), a printer assembly support (530) and a collector (540). In some embodiments, the printer assembly support (530) supports one or more printer heads and/or nozzles (550), optional vibration/oscillation units (560), and reservoirs (570) connected to the printer heads or nozzles. In some embodiments, a nozzle (550) that is used for electrospinning or electrospraying can be positively charged (e.g., with a high voltage of around 1-20 kV, for example, around 1 kV, 1-5 kV, around 5 kV, 5-10 kV, around 10 kV, 10-15 kV, around 15 kV, 15-20 kV, around 20 kV, or with a higher or lower voltage). In some embodiments, the collector (540) is grounded or slightly negatively charged. In some embodiments, the collector is a static structure (e.g., a static plate). In some embodiments, the collector is a movable structure (e.g., a movable plate, a rotatable drum or mandrel). In some embodiments, the collector is a drum (e.g., mandrel) that can be mechanically rotated. Accordingly, in some embodiments, a deposition system can include a motor configured and arranged for rotating a mandrel. In some embodiments, the different support structures, the collector, any associated pumps, motors, actuators, etc. can all be connected physically (e.g., by attachment to one or more parts of a chamber, housing or other system support). In some embodiments, components such as the pumps, motors, actuators, etc., can be connected (e.g., hard-wired, or wirelessly) to a controller (e.g., computer system) to control their operation.

In some, embodiments, a movable collector is connected or attached to a movable platform configured and arranged for adjusting or changing (e.g., manually or automatically via a controller) the position of the collector prior to or during polymer or fiber deposition. The platform may be attached to one or more motors to control movement of the platform in an appropriate direction, in some embodiments. In some embodiments, the gap distance between the tip of a nozzle and the surface of a collector is different depending on the type of deposition that is being used. For example, three different gap distances are illustrated in FIG. 5. In some embodiments, the smallest gap distance can be used for a printer head (e.g., a jet printer head). This distance can be on the order of several millimeters or less (e.g., up to 15 mm, up to 10 mm, up to 5 mm, up to 1 mm), for example. In some embodiments, the intermediate gap distance can be used for electrospraying a polymer or fiber solution or melt. This distance can be between 0.5 cm and 15 cm for example (e.g., 0.5-5 cm, 5-10 cm, or 10-15 cm, or more or less). In some embodiments, the largest gap distance can be used for electrospinning a polymer or fiber solution or melt. This distance can be between 1 cm and 30 cm for example (e.g., 5-10 cm, 10-15 cm, 15-20 cm, 20-25 cm, 25-30 cm, or greater). In some embodiments, the gap distance between the surface of the collector and the tips of one or more nozzles or printer heads is not fixed and can be adjusted. In some embodiments, this adjustment can occur during fiber or polymer deposition. This allows for the gap distance to be changed to remain optimal (or at least favorable) for different polymer types or concentrations that may be introduced during deposition. It should be appreciated that an optimal or favorable gap distance is dependent on the nature and concentration of a polymer or fiber solution or melt that is being deposited (e.g., via electrospinning, electrospraying, pressure spraying, or other technique).

Accordingly, in some embodiments a nozzle or printer head may be connected to a motor (e.g., a mechanical actuator) that can alter the gap distance to the collector surface. It should be appreciated that the configuration illustrated in FIG. 5 is non-limiting. For example, in some embodiments a system has a single nozzle that can be used for electrospinning, electrospraying, or both. In some embodiments, the position of the single nozzle relative to the support and/or the collector can be adjusted (e.g., before and during deposition). In some embodiments, a system has at least two nozzles (e.g., 2, 3, 4, 5, 6, 7, 8, 9, 10, or more). In some embodiments, each nozzle can be adapted for either electrospinning, electrospraying, or both. It should be appreciated that the inner and/or out diameters of a nozzle may be different depending on the type of solution that is being used and whether electrospraying or electrospinning is implemented. In some embodiments, a system includes at least one nozzle for electrospinning and/or electrospraying, and at least one printer head for printing. In some embodiments, the printer may be used for printing a polymeric material, for example, that is deposited on to a collector. The same or different materials may also be deposited through electrospraying or electrospinning or both to achieve different structural patterns in a scaffold. In some embodiments, at least one nozzle for pressure spraying (e.g., painting) also can be included. In some embodiments, the different nozzle tips and printer heads are set at different gap distances from the collector surface as described herein. However, in some embodiments, the gap distances can be adjusted, for example, by using individual motors (e.g., actuators) associated with each nozzle or printer head, or by using one or more 1 dimensional, 2 dimensional, or 3 dimensional (e.g., X, XY, or XYZ) motors that can be used to control the relative position of the support and/or collector.

It should be appreciated that in some embodiments nozzles and/or printer heads can be configured horizontally and/or vertically relative to the collector. It should also be appreciated that in some embodiments nozzles and/or printer heads can be configured to rotate (e.g., using an actuator or motor, for example that can be programmed or controlled, for example by a controller). In some embodiments, this can be useful to generate a twisted thread or fiber as described herein.

In some embodiments, the one or more nozzles and/or printer heads each are connected to a reservoir (560). Each reservoir can contain a polymer or fiber solution or melt. In some embodiments, the temperature of each reservoir can be controlled. In some embodiments, a pump (e.g., peristaltic pump, rotary displacement pump, etc.) can be connected to each reservoir to pump the solution or melt to the nozzle or printer head.

The functions of the different motors, pumps, actuators, high voltage elements, printers, etc., can be controlled and/or integrated by one or more controllers (520). For example, in some embodiments a combination of electrospun, electrosprayed, pressure sprayed, and/or printed material may be deposited on a collector (e.g., simultaneously or sequentially). In some embodiments, a high voltage is applied to the appropriate nozzles during electrospinning and/or electrospraying, but may be switched off during printing or other form of material deposition. However, it should be appreciated that different control algorithms may be used depending on the desired application. FIG. 5 shows the high-voltage power source and the controller outside chamber (500). However, it should be appreciated that one or both may be located within the chamber as aspects of the disclosure are not limited in this respect.

Any suitable materials may be used for the various components disclosed herein. In some embodiments, suitable materials include plastic, metals, glass, composites or polymeric material. In some embodiments, certain components are electrically inert (e.g., neutrally charged, made of an insulating material). However, it should be appreciated that certain components should be electrically conductive. For example, the mandrel should be electrically conductive. Accordingly, it can be made from a metallic material or it can be coated with a metallic material, for example a metal layer or sheet (e.g., an aluminum layer or sheet). In some embodiments, the mandrel is a composite comprising one or more electrically conductive materials, in which the electrically conductive materials are arranged or dispersed in a manner suitable for controlling the electrical conductivity of the mandrel.

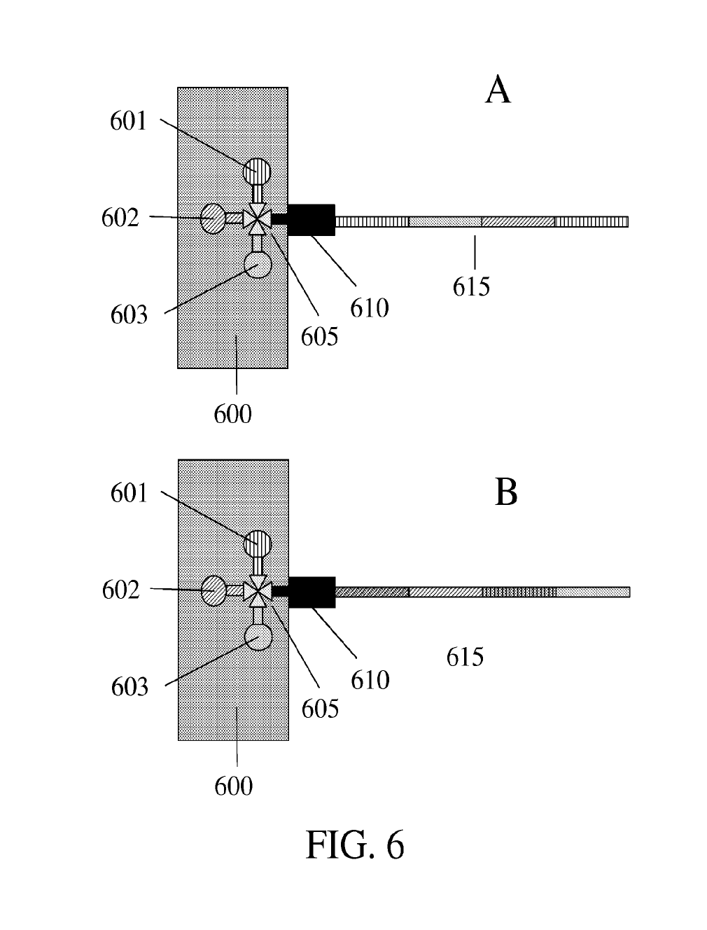

In some embodiments, one or more different polymer or fiber solutions or melts may be mixed during deposition. FIG. 6 illustrates a non-limiting embodiments wherein a reservoir support (600) supports three separate reservoirs (601, 602, and 603) that are connected to a valve (605) that is connected to a nozzle, printer head, or other dispenser (610). However, it should be appreciated that in some embodiments different numbers of separate reservoirs (e.g., 2, 3, 4, 5, 6, 7, 8, 9, 10 or more) can be connected to a valve or a network (e.g., a series) of valves in order to be able to control and vary the polymer or fiber solution or melt that is delivered to the collector surface. In some embodiments, a valve is a mixing valve. In some embodiments, a valve can control the relative amount of solution or melt from each of the reservoirs. In some embodiments, each reservoir is connected to a separate pump to pump the material into the mixing valve (and/or through the mixing valve to the nozzle or dispenser). In some embodiments, the mixing valve and/or the nozzle or dispenser is connected to a pump to pump the material through the nozzle or dispenser. It should be appreciated that in some embodiments the mixing valve and/or one or more pumps can be controlled by controller (520). In some embodiments, two or more nozzles, printer heads, or other dispensers (e.g., a described in connection with FIG. 5) each can be connected to two or more reservoirs via a mixing valve. In operation, the material from two or more different reservoirs can be mixed and/or alternated during deposition. FIG. 6A illustrates a non-limiting embodiment where alternating material is deposited. For example, the deposited fiber (615) illustrated in FIG. 6A includes material from reservoir 601 first, followed by material from reservoir 602, followed by material from reservoir 603, followed by material from reservoir 601. However, it should be appreciated that any order of material deposition from two or more different reservoirs can be produced. FIG. 6B illustrates a non-limiting embodiment wherein a mixture of different materials is deposited. For example, the deposited fiber (615) illustrated in FIG. 6B includes material from reservoir 603 first, followed by a mixture of material from reservoirs 601 and 603, followed by a mixture of material from reservoirs 602 and 603, followed by material from reservoir 601. However, it should be appreciated that different mixtures or combinations of mixtures may be produced.

It should be appreciated that the different mixtures in the different reservoirs can be selected to produce fibers having different properties (e.g., different relative elasticities, different degrees of cross-linking, different solubilities, and/or other different properties). Accordingly, in some embodiments one or more segmented and/or blended polymer or fiber flows can be created to have regions with different functional and/or structural properties. By changing the composition of the polymer or fiber solution or melt while it is being deposited, different segments of the same polymer or fiber deposition can having different physical properties (e.g., different elasticities, porosities, solubilities, conductivities, etc., or any combination thereof) can be created. These can impart macrostructure properties on a scaffold by providing different physical properties in different regions of the scaffold. This can be useful, for example, to incorporate one or more regions of relatively higher elasticity relative to other regions in a scaffold. In some embodiments, this can be useful to selectively introduce regions that can be dissolved from a scaffold (e.g., during seeding and/or after implantation) thereby producing a scaffold with a predetermined pattern of pores, cavities, or other internal structural shapes that can be useful to promote desired structural or functional properties (including, but not limited to, providing regions having different relative elasticity).

In some embodiments, a support (e.g., mandrel) may be heated while electrospinning is taking place so as to soften certain components to improve adhesion (e.g., to heat the larger solid diameter components, for example ribs of an airway scaffold so that the fibers stick better to the solid ribs). Accordingly, a heated support (or a support with different patterns of heat) can be used. In some embodiments, adhesion between the ribs and the fibers can also be enhanced by spraying solvent (e.g., hexafluoroisopropanol, or one or more other hexanes, or other solvents or adhesives) over the ribs prior to the deposition of fibers or by varying the amount of solvent present on the fibers at the point of deposition (which can be determined as a function of many variables including flow rate, polymer concentration, humidity etc.) so that the fibers that first contact the rib contain a higher level of solvent and so create chemical bonding between the fiber and the rib. In some embodiments, edges can be smoothed in order to reduce issues associated with sharp edges of certain portions of a scaffold (for example, problems arising from ribs that have sharp edges that can tear or degrade scaffold fibers over time, for example in the context of an airway scaffold where there can be issues due to the movement of the host neck after implantation). In some embodiments, methods for smoothing edges (e.g., chemically, with heat, with abrasion, etc., or any combination thereof) can be applied either before the electrospinning, electrospraying, or other deposition technique begins and/or during the process. In some embodiments, to reduce problems associated with excessive wearing due to ribs or other structures that can protrude beyond the plane of a scaffold (e.g., beyond the plane of the back wall of a scaffold, for example, at the tracheal/esophageal surface) the ribs or other structures can be aligned so that there is no protrusion. In some embodiments, dies and jigs can be used to set up a composite, a mandrel can be shaped (e.g. grooved) to accept the volume of a rib and to align it in place on the mandrel and keep it there during the spinning process (which involves rotational and shear forces that can dislodge the ribs), and/or a member can be used into which the ends of the ribs can fit (e.g., at right angles to the ribs) that runs along the length of the trachea on both sides of the esophageal wall along the joint between the esophageal wall and the tracheal walls. In some embodiments, these techniques not only align the ribs properly, but can blunt their ends and make them less mobile and less likely to protrude through the tracheal/esophageal wall.

It should be appreciated that different material (e.g., different fibers) can be used in methods and compositions described herein. In some embodiments, the material is biocompatible so that it can support cell growth. In some embodiments, the material is permanent (e.g., PET), semi-permanent (e.g., it persists for several years after implantation into the host, or rapidly degradable (e.g., it is resorbed within several months after implantation into the host).

In some embodiments, PET (polyethylene terephthalate (sometimes written poly(ethylene terephthalate)) is used. PET is a thermoplastic polymer resin of the polyester family. PET consists of polymerized units of the monomer ethylene terephthalate, with repeating C₁₀H₈O₄ units. Depending on its processing and thermal history, polyethylene terephthalate may exist both as an amorphous (transparent) and as a semi-crystalline polymer. The semicrystalline material might appear transparent (particle size <500 nm) or opaque and white (particle size up to a few microns) depending on its crystal structure and particle size. Its monomer (bis-β-hydroxyterephthalate) can be synthesized by the esterification reaction between terephthalic acid and ethylene glycol with water as a byproduct, or by transesterification reaction between ethylene glycol and dimethyl terephthalate with methanol as a byproduct. Polymerization is through a polycondensation reaction of the monomers (done immediately after esterification/transesterification) with water as the byproduct.

Methods of electrospinning PET and other fibers are known in the art. Electrospinning is a versatile technique that can be used to produce either randomly oriented or aligned fibers with essentially any chemistry and diameters ranging from nm scale (e.g., around 15 nm) to micron scale (e.g., around 10 microns).

However, it should be appreciated that other fibers may be used as aspects of the invention are not limited in this respect.

Also, it should be appreciated that different methods of depositing fibers can be used as aspects of the invention are not limited in this respect. In some embodiments, methods are described in the context of electrospun fibers. However, other techniques may be used, including “air laid” methods such as melt blowing, melt spinning, and gas jet fibrillation. In some embodiments, different gradients of fibers, deposition conditions, solvents, curing conditions, etc., or any combination thereof may be used to obtain patterns of fibers that result in an elastic scaffold or artificial tissue.

In some embodiments, appropriate fiber diameters, fiber length/aspect ratios, pore size, thicknesses, solidity, basis weight, also may be controlled to optimize elasticity while also preserving suitable properties for cellularization. For example, an appropriate pattern of fibers that is suitable for a desired elasticity should accommodate fiber densities that are sufficiently porous for cells. For example, pores of about 1-100 microns in diameter (e.g., about 10-90 microns, about 20-80 microns, or about 50 microns in diameter) are suitable for most cell types. These pores are larger than those required for water or air in a nanofiber material. In some embodiments, electrospun fibers having diameters ranging from about one to a few hundred nanometers are deposited to accommodate pore sizes of about 1-100 microns in between the fibers. In some embodiments, pores are created in the elastic scaffold by incorporating a gas or volatile liquid into the polymer solution before electrospinning, such that bubbles created by the gas or volatile liquid create pores during electrospinning. In some embodiments, the polymer or polymer solution contains solid granules of size 1-100 microns that are dissolved after electrospinning to create pores of size 1-100 microns.

In some embodiments, multiple layers of fibers (e.g., of the same type or of different types) are deposited as described herein. In some embodiments, suitable binders, bicomponent fibers having a sheath that melts at a lower temperature than core and used to adhere other fibers, intertwining of fibers during fabrication by impinging one fiber layer into another, or other techniques, or any combination thereof may be used to connect different fiber layers.

Different types of polymers can be used to form elastic scaffolds and/or elastic templates as described herein. Examples of polymer materials that can be used in some embodiments described herein include both addition polymer and condensation polymer materials such as polyolefin, polyacetal, polyamide, polyester, cellulose ether and ester, polyalkylene sulfide, polyarylene oxide, polysulfone, modified polysulfone polymers and mixtures thereof. In some embodiments, materials that fall within these generic classes include polyethylene, polypropylene, poly(vinylchloride), polymethylmethacrylate (and other acrylic resins), polystyrene, and copolymers thereof (including ABA type block copolymers), poly(vinylidene fluoride), poly(vinylidene chloride), polyvinylalcohol in various degrees of hydrolysis (87% to 99.5%) in crosslinked and non-crosslinked forms. Examples of addition polymers tend to be glassy (a T_{g} greater than room temperature). This is the case for polyvinylchloride and polymethylmethacrylate, polystyrene polymer compositions or alloys or low in crystallinity for polyvinylidene fluoride and polyvinylalcohol materials. One class of polyamide condensation polymers are nylon materials. The term “nylon” is a generic name for all long chain synthetic polyamides. Typically, nylon nomenclature includes a series of numbers such as in nylon-6,6 which indicates that the starting materials are a C₆ diamine and a C₆ diacid (the first digit indicating a C₆ diamine and the second digit indicating a C₆ dicarboxylic acid compound). Another nylon can be made by the polycondensation of epsilon caprolactam in the presence of a small amount of water. This reaction forms a nylon-6 (made from a cyclic lactam—also known as epsilon-aminocaproic acid) that is a linear polyamide. Further, nylon copolymers are also contemplated. Copolymers can be made by combining various diamine compounds, various diacid compounds and various cyclic lactam structures in a reaction mixture and then forming the nylon with randomly positioned monomeric materials in a polyamide structure. For example, a nylon 6,6-6,10 material is a nylon manufactured from hexamethylene diamine and a C₆ and a C₁₀ blend of diacids. A nylon 6-6,6-6,10 is a nylon manufactured by copolymerization of epsilonaminocaproic acid, hexamethylene diamine and a blend of a C₆ and a C₁₀ diacid material.

Block copolymers are also useful in certain embodiments described herein. With such copolymers the choice of solvent swelling agent is important. The selected solvent is such that both blocks were soluble in the solvent. One example is an ABA (styrene-EP-styrene) or AB (styrene-EP) polymer in methylene chloride solvent. If one component is not soluble in the solvent, it will form a gel. Examples of such block copolymers are Kraton type of styrene-b-butadiene and styrene-b-hydrogenated butadiene (ethylene propylene), Pebax type of ε-caprolactam-b-ethylene oxide, Sympatex polyester-b-ethylene oxide and polyurethanes of ethylene oxide and isocyanates.

In some embodiments, addition polymers can be used. Addition polymers like polyvinylidene fluoride, syndiotactic polystyrene, copolymer of vinylidene fluoride and hexafluoropropylene, polyvinyl alcohol, polyvinyl acetate, amorphous addition polymers, such as poly(acrylonitrile) and its copolymers with acrylic acid and methacrylates, polystyrene, poly(vinyl chloride) and its various copolymers, poly(methyl methacrylate) and its various copolymers, can be solution spun with relative ease because they are soluble at low pressures and temperatures. However, in some embodiments, highly crystalline polymer like polyethylene and polypropylene may require high temperature, high pressure solvent when solution spun. In some embodiments, electrostatic solution spinning is used to make nanofibers and microfibers.

Certain embodiments can be implemented using fibers made from different polymer materials. In some embodiments, small fibers with good adhesion properties can be made from such polymers like polyvinylidene chloride, polyvinyl alcohol and polymers and copolymers comprising various nylons such as nylon 6, nylon 4,6; nylon 6,6; nylon 6,10 and copolymers thereof. Excellent fibers can be made from PVDF, but to make sufficiently small fiber diameters requires chlorinated solvents. Nylon 6, Nylon 66 and Nylon 6,10 can be electrospun. However, solvents such as formic acid, m-cresol, tri-fluoroethanol, hexafluoro isopropanol are either difficult to handle or very expensive. Examples of solvents include water, ethanol, isopropanol, acetone and N-methylpyrrolidone due to their low toxicity. Polymers compatible with such solvent systems have been extensively evaluated. Fibers made from PVC, PVDC, polystyrene, polyacrylonitrile, PMMA, PVDF may require additional adhesion means to attain structural properties. Examples of alcohol soluble polyamides include Macromelt 6238, 6239, and 6900 from Henkel, Elvamide 8061 and 8063 from DuPont and SVP 637 and 651 from Shakespeare Monofilament Company. Another group of alcohol soluble polyamide is type 8 nylon, alkoxy alkyl modifies nylon 66 (Ref. Page 447, Nylon Plastics Handbook, Melvin Kohan ed. Hanser Publisher, New York, 1995). Examples of poly(vinyl alcohol) include PVA-217, 224 from Kuraray, Japan and Vinol 540 from Air Products and Chemical Company.

It should be appreciated that elastic scaffolds can be prepared under sterile conditions and/or sterilized after production so that they are suitable for cellularization. It should be appreciated that the types of cells that are used for cellularization will depend on the tissue type that is being produced. In some embodiments, one or more different tissue-specific (e.g., tissue-specific stem or progenitor) cells may be used. In some embodiments, different combinations of epithelial, endothelial, and/or structural cell types may be used to populate an elastic scaffold. In some embodiments, cells are selected to be compatible (e.g., histocompatible) with the host into which the scaffold is being transplanted. In some embodiments, one or more cell types that are isolated from the host are used to seed the scaffold. In some embodiments, the seeded scaffold is incubated to allow the cells to grow and further populate the scaffold prior to surgical implantation.

It should be appreciated that cell types used to seed a scaffold of the invention may be selected based on the type of structure (e.g., tissue, organ) that is being grown. In some embodiments, the cells may be epithelial, endothelial, mesothelial, connective tissue cells, fibroblasts, etc., or any combination thereof. In some embodiments, the cells may be stem cells, progenitor cells, mesenchymal stem cells, induced pluripotent stem cells, stromal cells, fibroblasts, chondrocytes, etc. These cells can be readily derived from appropriate organs or tissue such as skin, liver, blood, etc., using methods known in the art.

It should be appreciated that the number of cells required to cellularize an elastic scaffold as described herein will depend on the size of the scaffold, which will depend on the size of the tissue being replaced. It should be appreciated that techniques and material described herein can be used to produce any suitable size or shape of elastic scaffold (e.g., planar structures, tubular structures, hollow structures, solid structure, complex structures, any of which can have one or more dimensions ranging for example from about 1 mm to 50 cms (for example tracheal regions of several cms in length). However, larger, smaller, or intermediate sized structures may be made as described herein.

It should also be appreciated that elastic scaffolds may be seeded with cells using any of a variety of methods that permit cells to attach to the scaffold. For example, cells suspended in a medium (e.g., a cell culture medium) may be washed or poured over a scaffold for a sufficient duration and in sufficient quantities to permit cells to contact and attach to the scaffold. In some embodiments, a scaffold may be bathed in a cell culture bath to seed cells on the scaffold. In some embodiments, a scaffold may be rotated in a cell culture bath such that cells from the bath contact and attach to the scaffold. In some embodiments, the scaffold may be seeded uniformly. In some embodiments, cells are seeded non-uniformly over the scaffold, e.g., by pouring cells over one or more different regions of the scaffold. Additional methods for seeding cells on a scaffold are disclosed, for example, in United States Patent Application Publication No. 20110033918, entitled Rotating Bioreactors, the contents of which are incorporated herein by reference. In some embodiments, cells can be printed onto the surface of a scaffold (e.g., using a printer head of a system or device described herein). Suitable methods for printing cells are disclosed, for example, in United States Patent Application Publication No. 20110250688, entitled Three Dimensional Tissue Generation, the contents of which are incorporated herein by reference. In some embodiments, cells can be printed onto the surface of a scaffold using a ink-jet printer or a valve-based cell printer or other suitable printer. In some embodiments, a printer may be used to deliver cells suspended in a matrix, e.g., a hydrogel, or in a cell culture medium. In some embodiments, a printer may be used to deliver cells in a mixture with one or more other components, e.g., colloidal nanoparticles. In some embodiments, a single cell type may be delivered by a printer to a scaffold surface. In some embodiments, multiple different cell types may be delivered by a printer to a scaffold surface. In some embodiments, multiple different cell types may be delivered in a mixture. In some embodiments different cells types may be delivered in layers, the layers containing different cell types or different mixture of cell types.

In some embodiments, surface properties of the elastic scaffold can be modified either before seeding, during seeding, or after implantation. In some embodiments, the surface is hydrophilized by vacuum plasma surface activation. In some embodiments, vacuum plasma surface activation treatment is used to sterilize the elastic scaffold or to enhance cell attachment to the scaffold, or both. It should be appreciated that other techniques may be used to sterilize a scaffold prior to seeding with cells.

In some embodiments, different cells may be used to seed the outer and inner surfaces of a tubular structure (e.g., to form different inner and outer layers that correspond, at least in part, to natural inner and outer layers of a natural body structure). In some embodiments, only the inner or the outer surface of the support is seeded with cells. In some embodiments, the elastic scaffold undergoes a testing protocol before it is implanted into a host. In some embodiments, this protocol may include, but is not limited to, mechanical tests (e.g., torsional stress, non-symmetric elongation, transversal contraction, and long-term durability) and biological tests (e.g. cell attachment, cell viability, and sterility). For example, a test may be performed to confirm that a scaffold (e.g., prior to or after seeding) does not break at a 10% strain or more (e.g., at 15% strain, 20% strain, 25% strain, 50% strain, 75% strain, 100% strain or more, for example up to 150% strain, up to 200% strain or more), in at least one direction.

Embodiments of the present invention can be implemented in any of numerous ways. For example, some embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format. In some embodiments, the computer functions as a controller to control operation of one or more systems disclosed herein, e.g., a printer system, electrospinning system or electrospraying system.

Further, it should be appreciated that a computer may be embodied in any of a number of forms, such as a rack-mounted computer, a desktop computer, a laptop computer, or a tablet computer. Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format. In some embodiments, the output device of the computer is a printer for delivering cells, polymeric materials or other component to a scaffold or collector or other substrate in a particular pattern.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods, algorithms or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above.

As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (e.g., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements. While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A tissue scaffold that comprises one or more types of nanofibers and that is elastic in a first direction.

2. The tissue scaffold of claim 1, wherein the scaffold extends by 10-20% upon the application of about 5N of force in the first direction.

3. The tissue scaffold of claim 1, wherein the scaffold extends by 20-40% upon the application of more than about 20N of force in the first direction.

4. The tissue scaffold of claim 1, wherein the scaffold extends by 20-40% upon the application of more than about 40N of force in the first direction.

5. The tissue scaffold of claim 1, wherein the scaffold extends by 20-40% upon the application of more than about 60N of force in the first direction.

6. The tissue scaffold of claim 1, wherein the scaffold extends by 20-40% upon the application of more than about 80-100N of force in the first direction

7. The tissue scaffold of any prior claim, wherein the scaffold can extend by up to 100% in the first direction without experiencing structural failure.

8. The tissue scaffold of any prior claim, wherein the scaffold can extend by up to 150% in the first direction without experiencing structural failure.

9. The tissue scaffold of any prior claim, wherein the scaffold can extend by over 150% in the first direction without experiencing structural failure.

10. The tissue scaffold of any prior claim, wherein the one or more types of nanofiber include at least one electrospun nanofiber.

11. The tissue scaffold of claim 10, wherein the at least one electrospun nanofiber is PET.

12. The tissue scaffold of any prior claim, wherein the one or more types of nanofiber include a nanofiber having a diameter of about 10-500 nm.

13. The tissue scaffold of any prior claim, wherein the one or more types of nanofiber include a nanofiber having a diameter of about 200-400 nm.

14. The tissue scaffold of any prior claim, wherein the one or more types of nanofiber include a nanofiber having a diameter of about 300 nm.

15. The tissue scaffold of any prior claim, wherein the one or more types of nanofiber have a density that provides pore spaces of 1-100 microns.

16. The tissue scaffold of any prior claim, wherein the one or more types of nanofiber have a density that provides pore spaces of about 50 microns.

17. The tissue scaffold of any prior claim, wherein the scaffold is cellularized with one or more cell types.

18. The tissue scaffold of claim 17, wherein the one or more cell types are obtained from a host into which the scaffold is to be implanted.

19. The seeded scaffold of claim 17 or 18, wherein the one or more cell types are stem or progenitor cells.

20. The tissue scaffold of claim 18, wherein the host is a human host.

21. The tissue scaffold of any prior claim, wherein the scaffold is tubular.

22. The tissue scaffold of claim 21, wherein the scaffold has the shape and size of a human tracheal region.

23. The tissue scaffold of claim 22, wherein the scaffold is branched.

24. The tissue scaffold of any of claims 21-23, wherein the scaffold is elastic along the linear axis of the tubular shape.

25. A method of producing an elastic tissue scaffold, the method comprising depositing one or more nanofiber types on to an elastic template.

26. The method of claim 25, wherein the elastic template is manufactured from an elastic polymer.

27. The method of claim 25, wherein the elastic template is hollow.

28. The method of claim 25, wherein the elastic template is tubular.

29. The method of claim 25, wherein the elastic template is planar.

30. The method of claim 25, wherein the elastic template is shaped like a tissue, organ, or portion thereof.

31. The method of claim 25, wherein the one or more nanofiber types are deposited by electrospinning.

32. The method of claim 31, wherein the one or more nanofiber types include a PET nanofiber.

33. The method of claim 32, wherein the thickness of each of the one or more nanofiber types is between about 10 nm and about 500 nm.

34. The method of any of claims 25-33, wherein the one or more nanofiber types have a density that provides for pore sizes of about 1-100 microns.

35. A method of producing an elastic tissue scaffold, the method comprising depositing one or more nanofiber types on a solid support, wherein the one or more nanofiber types are deposited in a pattern that allows a plurality of nanofibers to move relative to each other to allow the scaffold to be stretched in at least one direction.

36. The method of claim 35, wherein the pattern is a woven pattern, a cross-hatched pattern, a net patterns, or other regular pattern of intersecting fibers.

37. The method of claim 35, wherein the solid support is shaped like a tissue, organ, or portion thereof.

38. The method of claim 35, wherein the one or more nanofiber types are deposited by electrospinning.

39. The method of claim 38, wherein the one or more nanofiber types include a PET nanofiber.

40. The method of claim 39, wherein the thickness of each of the one or more nanofiber types is between about 10 nm and about 500 nm.

41. The method of any of claims 25-40, wherein the one or more nanofiber types have a density that provides for pore sizes of about 1-100 microns.

42. A method of producing an elastic tissue scaffold, the method comprising depositing one or more nanofiber types on a solid support, wherein the one or more nanofiber types are deposited in a folded or coiled configuration that can be extended upon the application of a force, thereby allowing the scaffold to be stretched in at least one direction.

43. A method of producing an elastic tissue scaffold, the method comprising depositing one or more nanofiber types on a solid support under conditions to impart a curvature on the one or more nanofiber types, wherein the curvature can be straightened upon the application of a force, thereby allowing the scaffold to be stretched in at least one direction.

44. The method of claim 42 or 43, wherein the solid support is shaped like a tissue, organ, or portion thereof.

45. The method of claim 42 or 43, wherein the one or more nanofiber types are deposited by electrospinning.

46. The method of claim 45, wherein the one or more nanofiber types include a PET nanofiber.

47. The method of claim 46, wherein the thickness of each of the one or more nanofiber types is between about 10 nm and about 500 nm.

48. The method of any of claims 42-47, wherein the one or more nanofiber types have a density that provides for pore sizes of about 1-100 microns.

49. The method of any one of claims 25-48, further comprising sterilizing the elastic scaffold.

50. The method of any one of claims 25-49, further comprising cellularizing the elastic scaffold.

51. The method of any one of claims 25-50, further comprising implanting the elastic scaffold into a host.

52. The method of claim 51, wherein the host is an animal.

53. The method of 51, wherein the host is human.

54. The method of any one of claims 25-53, wherein a diseased or injured tissue is being replaced.

55. The method of any one of claims 25-53, wherein the diseased tissue is cancerous.

56. The tissue of any of claims 1-24, wherein the nanofibers are deposited by vibration of the support or nozzle, wherein the vibration is sufficient to create a nanofiber pattern.

57. The tissue of claim 56, wherein the nanofiber pattern is folded or wavy.

58. The tissue of claim 56 or 57, wherein the tissue can withstand greater than 10% strain without failure.

59. The tissue of claim 56-58, wherein the tissue can withstand greater than 20% strain without failure.

60. The tissue of claim 56-58, wherein the tissue can withstand greater than 30% strain without failure.

61. A device for generating a synthetic tissue scaffold, the device comprising
a collector;
an electrospray or electrospinning device configured and arranged for depositing a synthetic material on the collector; and
a printer device configured and arranged for depositing cells and/or a synthetic material on the collector.
